(19) United States
(12) Patent Application Publication
(10) Pub. No.: US 2011/0040149 A1
(43) Pub. Date: Feb. 17, 2011

(54) OBTURATOR ASSEMBLY

(76) Inventor: Robert C. Smith, Middletown, CT (US)

Correspondence Address: Tyco Healthcare Group LP;d/b/a Covidien, 555 Long Wharf Drive, Mail Stop 8-N1, Legal Department, New Haven, CT, 06511, US

(21) Appl. No.: 12/517,297

(22) PCT Filed: Jan. 11, 2008
(86) PCT No.: PCT/US08/00420

§ 371 Date: Jun. 2, 2009

Related U.S. Application Data
(60) Provisional application No. 60/880,164, filed on Jan. 12, 2007.

(51) Int. Cl. A61B 1/04

(52) U.S. Cl. 600/114

(57) ABSTRACT

An optical obturator for penetrating tissue includes an outer member defining a longitudinal axis and having proximal and distal ends, a leading member disposed adjacent the distal end of the outer member and having an optical window adapted to permit passage of light therethrough for detection by a clinician, and a penetrating member mounted adjacent the leading member and having a penetrating surface adapted to facilitate penetrating of tissue. The leading member is adapted for longitudinal movement between a first longitudinal position and a second longitudinal position. The penetrating surface of the penetrating member is at least partially exposed upon movement of the leading member from the first longitudinal position to the second longitudinal position.

### BACKGROUND

1. Technical Field

The present invention relates to an apparatus for the penetration of body tissue. More particularly, the present invention relates to an optical obturator with a retractable penetrating end.

2. Background of Related Art

In endoscopic surgical procedures, surgery is performed in any hollow viscus of the body through a small incision or through narrow endoscopic tubes (cannulas) inserted through a small entrance wound in the skin. In laparoscopic procedures, surgery is performed in the interior of the abdomen. Laparoscopic and endoscopic procedures often require the surgeon to act on organs, tissues and vessels far removed from the incision, thereby requiring that any instruments used in such procedures be of sufficient size and length to permit remote operation.

In laparoscopic procedures, in general, the surgical region is first insufflated. Thereafter, a trocar assembly, generally including a cannula and a stylet or obturator having a sharp tip for penetrating the body cavity, is typically used to create percutaneous access. Following puncture the cannula will remain in place during the procedure, providing access for additional instrumentation. An example of a known trocar is described in commonly assigned U.S. Pat. No. 6,319,266 to Stellon, which issued Nov. 21, 2001, the contents of which are incorporated herein in its entirety by reference. With known trocars and trocar assemblies, advancement of the obturator through tissue is typically performed blind, that is, without visualization of the tissue being penetrated. Additionally, at present, most currently used trocars rely on protective tubes or relative retraction of the tip to prevent inadvertent contact with tissue.

### SUMMARY

The present disclosure relates to improvements in accessing and penetrating body tissue during endoscopic procedures, laparoscopic procedures and the like, disclosing an apparatus and a method of use thereof.

In one embodiment, an optical obturator for penetrating tissue includes an outer member defining a longitudinal axis and having proximal and distal ends, a leading member disposed adjacent the distal end of the outer member and having an optical window adapted to permit passage of light therethrough for detection by a clinician, and a penetrating member mounted adjacent the leading member and having a penetrating surface adapted to facilitate penetrating of tissue. The leading member is adapted for longitudinal movement between a first longitudinal position and a second longitudinal position. The penetrating surface of the penetrating member is at least partially exposed upon movement of the leading member from the first longitudinal position to the second longitudinal position. The first longitudinal position of the leading member may correspond to an advanced position relative to the outer member, and the second longitudinal position of the leading member may correspond to a retracted position relative to the outer member. In this arrangement, the leading member is adapted to move from the first longitudinal position to the second longitudinal position upon engagement with tissue during passage of the leading member through the tissue. The leading member may be normally biased toward the first longitudinal position thereof. A biasing member may be adapted to operatively engage the leading member to normally bias the leading member toward the first longitudinal position thereof. The biasing member may include a spring member.

The penetrating member is operatively connected to the outer member. The leading member may include a slot dimensioned to at least partially receive the penetrating member. The penetrating surface of the penetrating member is substantially confined within the slot when the leading member is in the first longitudinal position thereof and is at least partially exposed from the slot when the leading member is in the second longitudinal position thereof. The penetrating member may include a bladed knife.

The outer member preferably includes a longitudinal opening adapted for reception of an endoscope. Alternatively, an imaging element may be associated with the outer member and adapted to transmit an image received through the optical window.

The leading member may define an arcuate configuration including, e.g., a general semi-hemispherical configuration.

In another embodiment, an optical obturator includes an outer sleeve member defining a longitudinal axis, and having proximal and distal ends and a longitudinal opening for reception of an endoscope, an optical member disposed adjacent the distal end of the outer sleeve member and adapted to transfer an image of an object for detection by the endoscope, and a penetrating member operatively connected to the outer sleeve and being at least partially disposed within the optical member. The optical member is adapted for longitudinal movement from an advanced position to a retracted position upon engagement thereof with tissue during entry of the optical member through tissue. The penetrating member includes a penetrating surface adapted to penetrate through tissue. The penetrating surface is at least partially exposed from the optical member when the optical member is in the retracted position thereof.

The penetrating member may be longitudinally fixed relative to the outer sleeve member. The optical member is adapted for reciprocal longitudinal movement relative to the penetrating member. A biasing member may be adapted to normally bias the optical member toward the advanced position thereof.

The optical member may include a slot adapted for at least partial reception of the penetrating member. The optical member defines a general hemispherical-shaped configuration. The penetrating member may be a knife blade having a piercing surface adapted to pierce tissue.

### BRIEF DESCRIPTION OF THE DRAWINGS

The accompanying drawings, which are incorporated in, and constitute a part of this specification, illustrate embodiments of the disclosure and, together with a general description of the disclosure given above and the detailed description of the embodiment(s) given below, serve to explain the principles of the disclosure, wherein:

FIG. 1 is a perspective view of a surgical system in accordance with the principles of the present disclosure illustrating the optical obturator, an endoscope for insertion within the optical obturator, and a cannula assembly;

FIG. 2 is a perspective view of the optical obturator of the surgical system;

FIG. 3 is a side plan of the optical obturator of FIGS. 1-2 in an initial advanced longitudinal position;

FIG. 4 is a cross-sectional view of the optical obturator in the initial advanced position and taken along the lines 4-4 of FIG. 3;

FIG. 5 is an axial view of the optical obturator in the initial advanced position;

FIG. 6 is a side plan view of the optical obturator in a retracted longitudinal position;

FIG. 7 is a cross-sectional view of the optical obturator in the retracted position;

FIG. 8 is a view illustrating the optical obturator mounted to the cannula assembly, with the endoscope positioned therein, to permit visualization during penetration of tissue; and

FIG. 9 illustrates a methodology for using the apparatus of FIG. 1-8 in accordance with the principles of the present invention.

### DESCRIPTION OF EMBODIMENTS

Specific embodiments of the presently disclosed apparatus and method will now be described in detail with reference to the foregoing figures wherein like reference numerals identify similar or identical elements. In the figures and in the description which follows, the term “proximal”, as is traditional will refer to the end of the apparatus or instrument of the present disclosure which is closest to the clinician, while the term “distal” will refer to the end of the device or instrument which is furthest from the clinician. In addition, the term “transparent” is to be interpreted as describing the ability to permit the passage of light with or without clear imaging capabilities. Moreover, any reference to any transparent material, or to any material that may be as transparent, includes any transparent or translucent material or any material which is not opaque to visible light or other radiation utilized for imaging purposes.

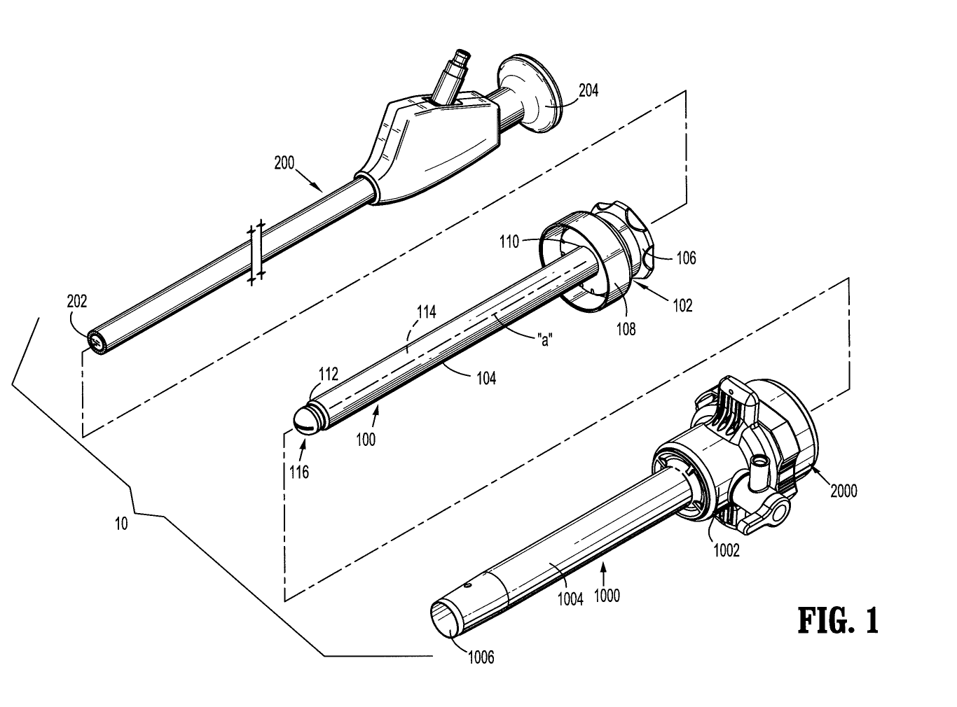

Referring now to the drawings, FIG. 1 illustrates a surgical system in accordance with the present disclosure. System 10 has particular application in laparoscopic procedures with respect to accessing the abdominal cavity, and the like, and includes optical obturator 100, endoscope 200 and cannula assembly 1000, In general, endoscope 200 is at least partially positioned within optical obturator 100, and the assembled unit is received within cannula assembly 1000. The system 10 is applied against the abdominal wall whereby optical obturator 100 punctures or penetrates the abdominal cavity under direct visualization via endoscope 200, thereby providing visual confirmation of entry into the body cavity while also substantially minimizing any undesired contact or engagement with any underlying organs. Obturator 100 and endoscope 200 are then removed from cannula assembly 1000 to permit the subsequent introduction of surgical instrumentation utilized to carry out the remainder of the procedure through cannula assembly 1000. As an alternative, endoscope 200 may be positioned within optical obturator 100 after the optical obturator 100 has been inserted into the body cavity through cannula assembly 1000.

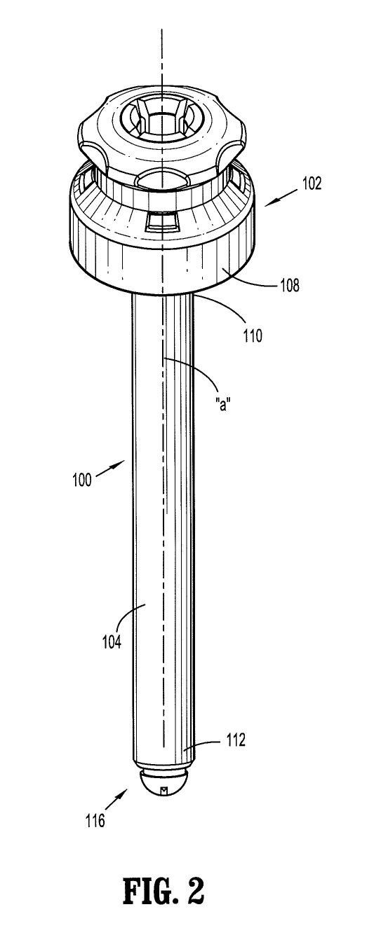

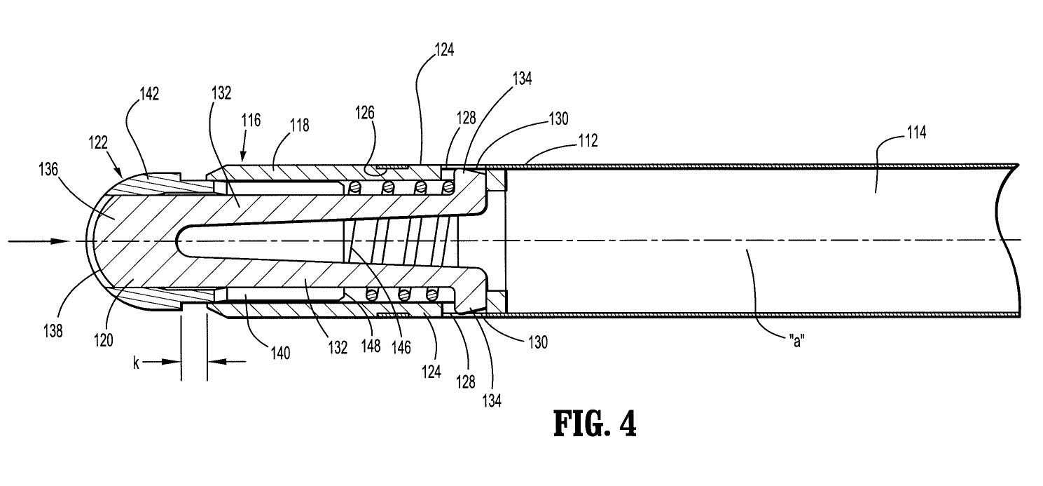

With reference to FIGS. 2-5, in conjunction with FIG. 1, optical obturator 100 includes obturator housing 102 and sleeve or outer member 104 extending from the housing 102.

Housing 102 is advantageously dimensioned for grasping by the clinician. In one embodiment, housing 102 may include locking collet 106 to secure endoscope 200 within optical obturator 100 in, e.g., a similar manner as described in commonly assigned U.S. patent application Ser. No. 11/103892 to Smith, the entire contents of which are hereby incorporated by reference. Housing 102 may further define skirt 108 which mates with corresponding structure of cannula assembly 1000. Outer member 104 defines proximal or trailing end 110 and leading or distal end 112. Outer member 100 further defines longitudinal axis “a” and has longitudinal lumen 114 extending at least partially along the length of outer member 104. Housing 102 and outer member 104 may be fabricated from any suitable biocompatible metal such as stainless steel and titanium and its alloys. Alternatively, these components may include a polymeric material such as polycarbonate, polystyrene, etc. and may be manufactured through known molding.

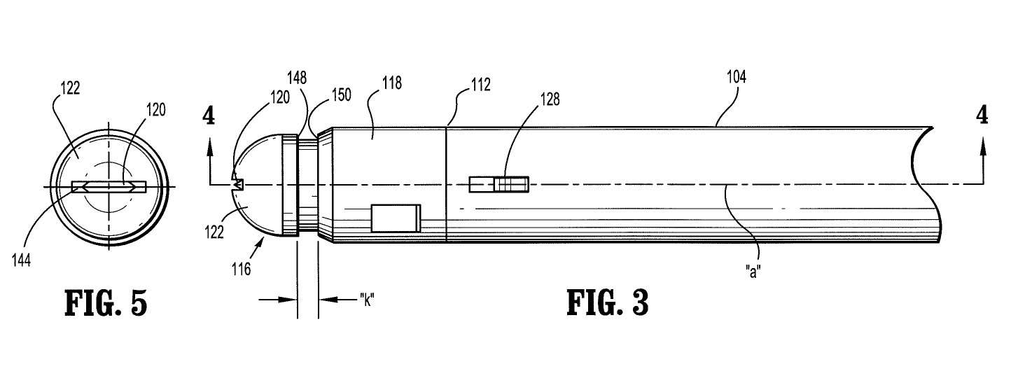

Referring now to FIGS. 3-5, optical obturator 100 further includes penetrating end 116 adjacent leading or distal end 112 of outer member 104. Penetrating end 116 incorporates penetrating housing 118, penetrating member 120 secured within the penetrating housing 118 and transparent or optical member 122 disposed about the penetrating member 120. Penetrating housing 118 is preferably secured to outer member 104 by conventional means. In one embodiment, penetrating housing 118 includes a pair of locking detents or ribs 124 adjacent corresponding recesses 126 within the penetrating housing 118. Locking ribs 124 are adapted for reception within corresponding locking openings 128 in outer member 104 in snap relation therewith to secure penetrating housing 118 to the outer member 104. In the alternative, penetrating housing 118 may be secured to outer member 104 through other means including adhesives, cements, screw threading etc. As a further embodiment, penetrating housing 118 may be integrally or monolithically formed within outer member 104. Penetrating housing 118 also defines a pair of openings 130 adjacent its proximal end adapted for mounting penetrating member 120 within the penetrating housing 118 as will be discussed,

Penetrating member 120 may be any suitable element adapted to penetrate and/or pierce tissue including, e.g., a pyramidal or sharpened conical member, and may or may not incorporate sharpened edges or surfaces. In one embodiment, penetrating member 120 is a planar bladed element secured within penetrating housing 118 and at least partially extending beyond outer member 104. In particular, penetrating member 120 includes proximal legs 132 having locking detents 134 extending radially outwardly from the legs 132. Locking detents 134 are dimensioned to be received within openings 130 of penetrating housing 118 in snap relation to secure the penetrating member 120 relative to outer member 104. Specifically, proximal legs 132 may be moved radially inwardly during insertion within penetrating housing 118 to permit passage through the penetrating housing 118 and then released whereby locking detents 134 are received within openings 130. With this arrangement, penetrating member 120 is generally axially fixed relative to outer member 104. In the alternative, penetrating member 120 may be capable of longitudinal movement relative to outer member 104.

Penetrating member 120 further includes elongated blade portion 136 defining a generally arcuate outer surface 138. Outer surface 138 may be sharpened to facilitate piercing through tissue or, alternatively, may be blunt or atraumatic to be devoid of piercing capabilities.

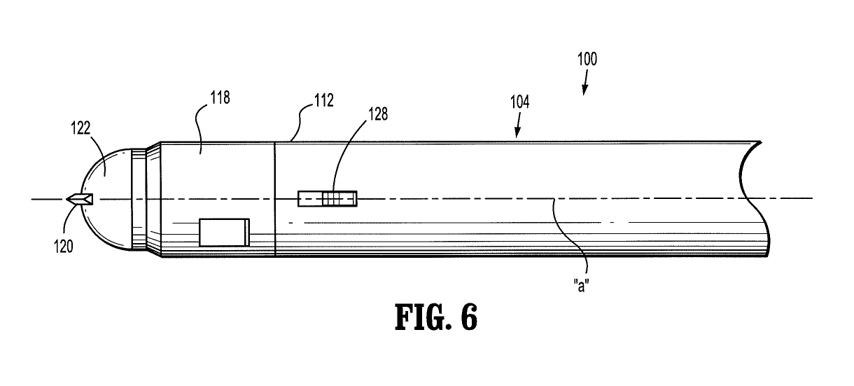

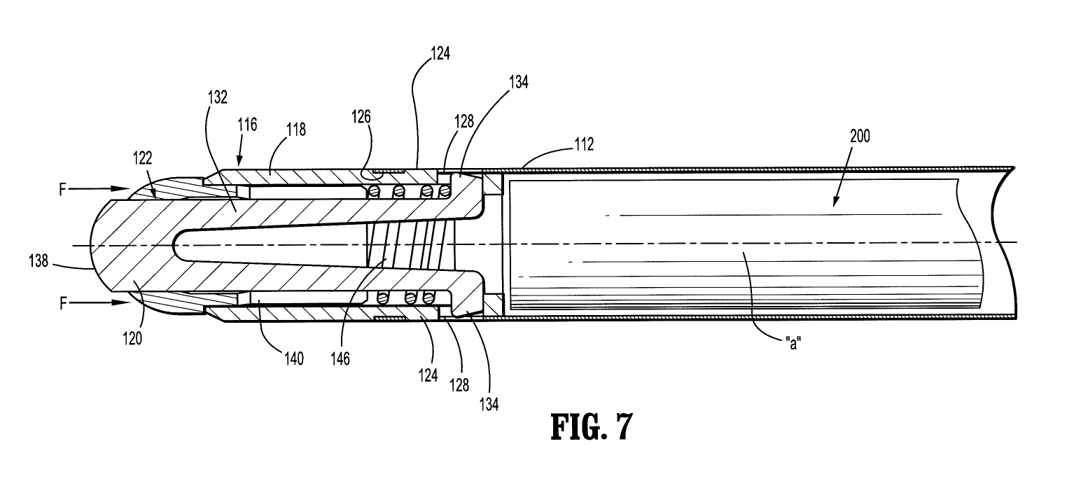

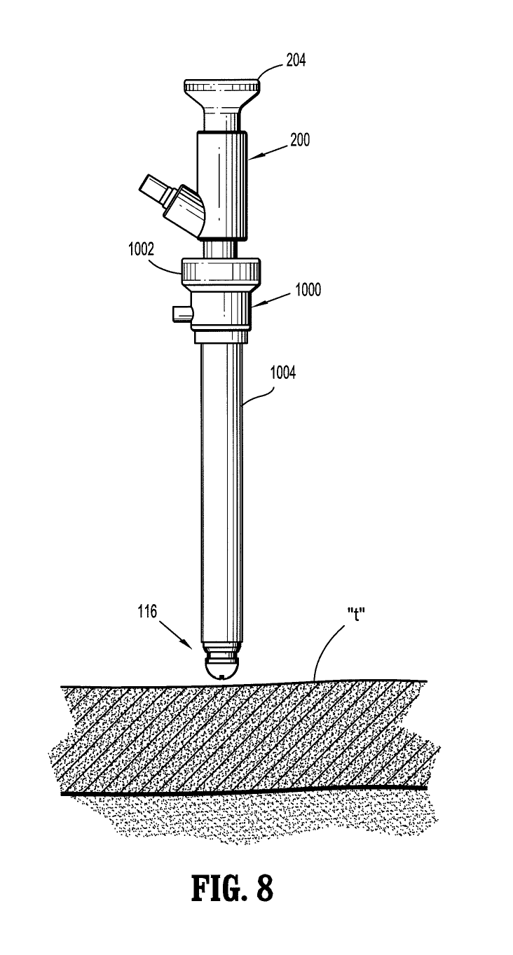

Referring now to FIGS. 3-7, optical member 122 is mounted within penetrating housing 118 and is preferably adapted for reciprocal longitudinal movement relative to the penetrating housing 118 and penetrating member 120 between an initial or first advanced longitudinal position depicted in FIGS. 3-5 and a second or retracted longitudinal position depicted in FIGS. 6-7. Optical member 122 includes proximal cylindrical portion 140 which is received within penetrating housing 118 and optical window or dome 142 extending from the cylindrical portion 140. Any means for mounting optical member 122 within penetrating housing 118 are envisioned. Optical member 122 includes longitudinal slot 144 (FIG. 5) which bisects the optical member 124 and is adapted for reception of penetrating member 120. Optical member 122 is distally biased toward the first longitudinal position by biasing member 146. In one embodiment, biasing member 146 is a coil spring which, at its proximal end, engages locking detents 134 of penetrating member 120 and, at its distal end, engages proximal end 148 of optical member 122. In the first longitudinal position of optical member 122 depicted in FIGS. 3-5, penetrating member 122 is preferably contained within the outer boundary of the optical member 122 thus avoiding any undesired contact of the penetrating member 122 with the clinician or tissue. In the second retracted position of optical member 120 depicted in FIGS. 6 and 7 as effected through a proximal force “F” (e.g., due to engagement with tissue) on the optical member 120, the penetrating member 120 is exposed to sever, incise, or penetrate tissue.

Optical dome 146 is preferably transparent at least in part or defines a window to permit transmission of light and/or of an image. In one embodiment, optical dome 146 is generally semi-hemispherical in shape. Other configurations are also envisioned including conical, ogive, pyramidal etc. Optical dome 146 defines circumferential ledge 148. Ledge 148 is adapted to engage distal end 150 of penetrating housing 118 upon movement to the second retracted longitudinal position thereby providing control of the degree of retraction of optical member 122. Preferably, the distance or spacing “k” between ledge 148 and distal end 150 of penetrating housing 118 is predetermined to permit sufficient exposure of outer surface 138 of penetrating member 120 to facilitate penetration through tissue.

Referring again to FIG. 1, endoscope 200 may be any conventional scope suitable for endoscopic applications including, e.g., a laparoscope, arthroscope, colonoscope, etc. In one embodiment, endoscope 200 may be the scope disclosed in commonly assigned U.S. Patent No. 5,412,504 to Leiner (hereinafter “Leiner”), the entire contents of which are hereby incorporated by reference. Endoscope 200 incorporates an optical train or lens arrangement capable of transmitting an image from distal window 202 to eye piece 204 for viewing by the surgeon and may incorporate an illuminating system for providing light. Although FIG. 1 depicts endoscope 200 with eye piece 204, it is also contemplated that endoscope 200 may, additionally or alternatively, be connected to a monitor. Further details regarding endoscope 200 may be ascertained by reference to Leiner.

The present disclosure also contemplates that the optical obturator 100 may be fitted with an internal or integral illumination or imaging system thereby avoiding the need for endoscope 200, i.e., the illumination and imaging system would be built into optical obturator 100. Those skilled in the art would appreciate the manner in which to modify optical obturator 100 to incorporate an internal illumination or imaging system, into a single unit.

Referring again to FIG. 1, cannula assembly 1000 of the system 10 will now be discussed. Cannula assembly 1000 may be any cannula assembly suitable for the purpose of accessing a body cavity. As an example, the apparatus of the present disclosure may be used in a laparoscopic surgical procedure where the peritoneal cavity is insufflated with a suitable gas, e.g., CO₂, to separate the cavity wall from the internal organs housed therein. In one embodiment, cannula assembly 1000 includes cannula housing 1002 with cannula sleeve 1004 extending therefrom. Either or both of cannula housing 1002 and cannula sleeve 1004 may be opaque or transparent, either wholly or in part, and may be fabricated from any biocompatible material including metals or polymers. Cannula sleeve 1004 defines an internal longitudinal lumen 1006 dimensioned to permit the passage of surgical instrumentation. It is contemplated that the diameter of cannula sleeve 1004 may vary in diameter up to 15 mm, or larger, dependent upon the procedure in which it is employed and the corresponding size of the instrument to be inserted therein. Cannula assembly 1000 may include an internal seal or valve (not shown), such as a duck-bill valve or other zero closure valve, adapted to close in the absence of a surgical instrument to prevent passage of insufflation gases through the cannula assembly 1000, as is known in the art. An example of such an internal seal or valve is disclosed in commonly assigned U.S. Pat. No. 5,820,600 to Carlson, et. al., the disclosure of which is incorporated by reference herein.

Cannula assembly 1000 may also include a seal assembly 2000 which may be releasably mounted to cannula housing 1002. Means for releasably connecting seal assembly 2000 to cannula housing 1002 may include a bayonet coupling, threaded connection, latch, friction fit, tongue and groove arrangements, snap-fit, etc. Seal assembly 2000 includes at least one internal seal or valve (not shown) adapted to form a fluid tight seal about an instrument inserted therethrough, as is known in the art. An example of one such suitable seal is the fabric seal disclosed in commonly assigned U.S. Pat. No. 6,702,787 to Racenet et al. (hereinafter “Racenet”), the entire contents of which are incorporated herein by reference. The seal disclosed in the Racenet '787 patent may be a flat septum seal having a first layer of resilient material and a second fabric layer juxtaposed relative to the first layer. Further details of the seal may be ascertained by reference to Racenet. It is contemplated that seal assembly 2000 may or may not be a component of cannula assembly 1000. For example, it is contemplated that seal assembly may be a separate, removable assembly. In the alternative, the seal assembly may comprise an integral part of the cannula assembly 1000, therefore not being removable.

Referring to FIGS. 1, 7 and 8, the use and function of the system 10 will now be discussed. The peritoneal cavity is first insufflated with a suitable biocompatible gas such as, e.g., CO₂ gas, such that the cavity wall is raised and lifted away from the internal organs and tissue housed therein, providing greater access thereto. The insufflation may be performed with an insufflation needle or similar device, as is conventional in the art. Following insufflation, endoscope 200 is positioned within optical obturator 100, specifically, first through locking collet 106, then passed through longitudinal lumen 114 of outer member 104 and advanced such that distal window 202 of endoscope 200 is adjacent distal end 112 of outer member 104 specifically, adjacent penetrating end 116. FIG. 7 depicts endoscope 200 positioned within optical obturator 100. However, in FIG. 7, cannula 1000 is not shown. Endoscope 200 may be secured within optical obturator 100 through collet 106. Thereafter, optical obturator 100 and endoscope 200 are positioned within cannula assembly 1000 and advanced whereby skirt 108 mates with seal assembly 2000, or if cannula assembly 1000 is devoid of seal assembly 2000, the skirt 108 will mate with cannula housing 1002. The present disclosure also contemplates that endoscope 200 may be positioned within optical member 100 following the insertion of the optical obturator 100 into cannula assembly 1000.

With the system 10 fully assembled, the targeted tissue is penetrated. With reference to FIG. 8, penetrating end 116 is applied against the tissue “t”. As optical member 122 engages the tissue “t”, the optical member moves from the first longitudinal position depicted in FIGS. 3-5 to the second longitudinal position depicted in FIGS. 6-7 by the force “F” applied by the tissue against the bias of coil spring 146. In this condition, penetrating member 120 is at least partially exposed whereby outer surface 138 penetrates tissue. During penetration, endoscope 200 permits constant visualization of any neighboring, underlying or surrounding tissue during the distal advancement of penetrating end 116 of optical obturator 100. This allows the clinician to confirm entry into the body cavity while also providing a way to monitor the procedure, thereby insuring that underlying tissue and organs do not engage or come into contact with the penetrating member 122 of penetrating end 116. In instances where a video system is utilized, the surgeon simply observes the penetration of body tissue “t” via any known video monitor. Once the penetrating end 116 passes through tissue, optical member 122 is no longer constrained by forces applied by the tissue and is free to move to the first longitudinal position of FIGS. 3-5 under the influence of coil spring 146 to cover penetrating member 120. Optical obturator 100 and endoscope 200 may then be removed from cannula assembly 1000 to permit the introduction of other instruments to perform the clinical surgical procedure.

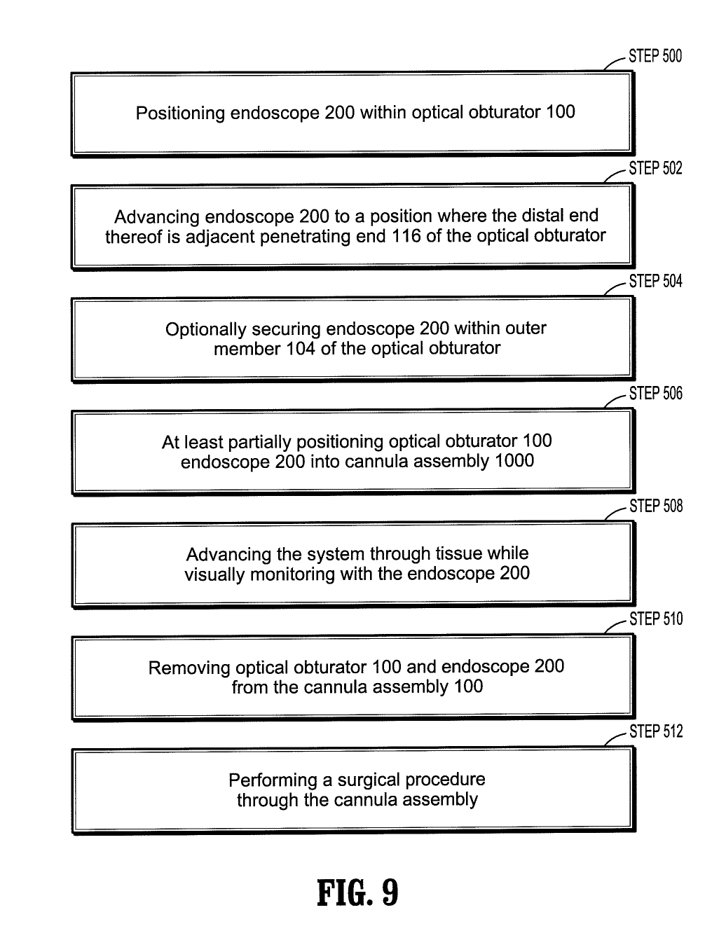

FIG. 9 illustrates a methodology for performing a surgical procedure according to the principles of the present disclosure. The method incorporates the steps of:

1) positioning an endoscope 200 within optical obturator 100 (STEP 500);

2) advancing endoscope 200 to a position where the distal end thereof is adjacent penetrating end 116 of the optical obturator (STEP 502);

3) Optionally securing endoscope 200 within outer member 104 of the optical obturator (STEP 504);

4) at least partially positioning optical obturator 100 endoscope 200 into cannula assembly 1000 (STEP 506);

5) advancing the system through tissue while visually monitoring with the endoscope 200 (STEP 508);

6) removing optical obturator 100 and endoscope 200 from the cannula assembly 100 (STEP 510); and

7) performing a surgical procedure through the cannula assembly (STEP 512).

While the above is a complete description of the embodiments of the present disclosure, various alternatives, modifications and equivalents may be used. Therefore, the above description should not be construed as limiting, but rather as illustrative of the principles of the disclosure made herein. Those skilled in the art will envision other modifications within the scope and spirit of the claims appended hereto.

What is claimed is:

1. An optical obturator for penetrating tissue, which comprises:
an outer member defining a longitudinal axis, and having proximal and distal ends;
a leading member disposed adjacent the distal end of the outer member and having an optical window adapted to permit passage of light therethrough for detection by a clinician, the leading member being adapted for longitudinal movement between a first longitudinal position and a second longitudinal position; and
a penetrating member mounted adjacent the leading member and having a penetrating surface adapted to facilitate penetrating of tissue, the penetrating surface at least partially exposed upon movement of the leading member from the first longitudinal position to the second longitudinal position.

2. The optical obturator according to claim 1 wherein the first longitudinal position of the leading member corresponds to an advanced position relative to the outer member and the second longitudinal position of the leading member corresponds to a retracted position relative to the outer member, the leading member adapted to move from the first longitudinal position to the second longitudinal position upon engagement with tissue during passage of the leading member through the tissue.

3. The optical obturator according to claim 2 wherein the leading member is normally biased toward the first longitudinal position thereof.

4. The optical obturator according to claim 3 including a biasing member adapted to operatively engage the leading member to normally bias the leading member toward the first longitudinal position thereof.

5. The optical obturator according to claim 4 wherein the biasing member includes a spring member.

6. The optical obturator according to claim 2 wherein the penetrating member is operatively connected to the outer member.

7. The optical obturator according to claim 6 wherein the leading member includes a slot dimensioned to at least partially receive the penetrating member.

8. The optical obturator according to claim 7 wherein the penetrating surface of the penetrating member is substantially confined within the slot when the leading member is in the first longitudinal position thereof and is at least partially exposed from the slot when the leading member is in the second longitudinal position thereof.

9. The optical obturator according to claim 8 wherein the penetrating member includes a bladed knife.

10. The optical obturator according to claim 2 wherein the outer member includes a longitudinal opening adapted for reception of an endoscope.

11. The optical obturator according to claim 2 including an imaging element associated with the outer member, the imaging device adapted to transmit an image received through the optical window.

12. The optical obturator according to claim 2 wherein the leading member defines an arcuate configuration.

13. The optical obturator according to claim 12 wherein the leading member defines a general semi-hemispherical configuration.

14. An optical obturator, which comprises:
an outer sleeve member defining a longitudinal axis, and having proximal and distal ends, the outer sleeve member including a longitudinal opening for reception of an endoscope;
an optical member disposed adjacent the distal end of the outer sleeve member, the optical member adapted to transfer an image of an object for detection by the endoscope, the optical member adapted for longitudinal movement from an advanced position to a retracted position upon engagement thereof with tissue during entry of the optical member through tissue; and
a penetrating member operatively connected to the outer sleeve and being at least partially disposed within the optical member, the penetrating member including a penetrating surface adapted to penetrate through tissue, the penetrating surface at least partially exposed from the optical member when the optical member is in the retracted position thereof.

15. The optical obturator according to claim 14 wherein the penetrating member is longitudinally fixed relative to the outer sleeve member.

16. The optical obturator according to claim 15 wherein the optical member is adapted for reciprocal longitudinal movement relative to the penetrating member.

17. The optical obturator according to claim 16 including a biasing member adapted to normally bias the optical member toward the advanced position thereof.

18. The optical obturator according to claim 16 wherein the optical member includes a slot adapted for at least partial reception of the penetrating member.

19. The optical obturator according to claim 18 wherein the penetrating member includes a knife blade having a piercing surface adapted to pierce tissue.

20. The optical obturator according to claim 14 wherein the optical member defines a general hemispherical-shaped configuration.
